SEQUENCE LISTING (2 sequences)

SEQ ID NO: 1 - DNA, 17 bp, Artificial Sequence
  primer rRNA16s forward
  misc_feature (9)..(9): n is a, c, g, or t
cctacgggnggcwgcag

SEQ ID NO: 2 - DNA, 21 bp, Artificial Sequence
  primer rRNA16s reverse
gactachvgggtatctaatcc

(19) United States
(12) Patent Application Publication
(10) Pub. No.: US 2020/0270675 A1
(43) Pub. Date: Aug. 27, 2020

(54) METHOD FOR EVALUATING THE STATE OF HEALTH OF AN INDIVIDUAL

(71) Applicant: WELLMICRO S.R.L., Bologna (IT)

(72) Inventors: Elena Biagi, Bologna (IT); Simone Rampell, Ferrara (IT); Silvia Turroni, Forli (IT); Andrea Castagnetti, Bologna (IT); Marco Candela, Bologna (IT)

(21) Appl. No.: 16/067,963

(22) PCT Filed: Jan. 4, 2017
(86) PCT No.: PCT/IB2017/050019

§ 371 Date: Jul. 3, 2018

(30) Foreign Application Priority Data
Jan. 5, 2016 (IT) 102016000000558

(51) Int. Cl. C12Q 1/689; G16B 40/00

(52) U.S. Cl. CPC C12Q 1/689; C12Q 2531/113; G16B 40/00

(57) ABSTRACT

The present invention relates to a method for determining and/or monitoring the state of health and/or balance of the intestinal microbiota of an individual, said method being characterised by the steps of: typing the bacterial populations present in a sample isolated from an individual, preferably a faecal sample, said populations being representative of the intestinal microbiota of the individual; and measuring a series of indices of the microbiota which provide information on the individual's state of health. The indices are the diversity index, the index of anti-inflammatory and/or immunomodulating potential, the index of association between said intestinal microbiota and at least one pathological condition, and the index of association between said intestinal microbiota and systemic aging. All the indices are calculated compared to a reference microbiota consisting of that of a population of healthy individuals.

The present invention relates to a method for determining and/or monitoring the state of health and/or balance of the intestinal microbiota of an individual which is based on: typing the bacterial populations present in a sample isolated from an individual, preferably a faecal sample, said populations being representative of the intestinal microbiota of the individual; and measuring a series of indices of the microbiota which provide information on the individual's state of health.

### BACKGROUND

At present, in order to be able to derive information on the state of health of an individual through an analysis of his or her intestinal microbiota, use is made essentially of methods based on classical microbiology (culture-dependent) or also “targeted” molecular methods, such as quantitative PCR. However, these methods aim to determine the presence of only a very limited number of specific pathogenic and/or non-pathogenic bacteria that are well known to populate the human intestine. In conclusion, the methods currently used do not enable a wide-spectrum analysis of the entire intestinal microbiota.

The characterisation of the entire intestinal microbiota is rapidly becoming indispensable in various areas of clinical research, such as, for example, gastroenterology, immunology, oncology and general medicine. The available literature indicates that a microbial profile (expressed in terms of relative abundance of the taxonomic units present inside the gastrointestinal tract) similar to the average profile obtainable from healthy individuals is an index associated/associable with the state of health and wellbeing of the individual concerned. In fact, a healthy and balanced composition of the intestinal microbiota is helpful, or sometimes indispensable, for maintaining good metabolic efficiency, a correct functioning of the immune system and a good level of prevention against disorders and pathologies such as, for example, obesity, diabetes, allergies, inflammatory intestinal diseases and pathologies tied to aging, which are increasingly frequent, particularly in the western population.

From the foregoing, it may be deduced that if one were able to evaluate the state of health of the entire intestinal microbiota of an individual, it would be possible to preserve or in any event to improve the balance thereof by adopting appropriate changes in the individual's diet and/or lifestyle. Preserving or improving the balance of the intestinal microbiota means maintaining and/or recovering an individual's state of health. In other words, if one were able to develop systems for monitoring, and thus per maintaining and/or recovering the state of health of an individual's intestinal microbiota, it would be possible to devise personalised dietetic and/or therapeutic approaches for preventing pathologies and/or in any case resolving pathological disorders, with obvious virtuous effects on public health care costs.

The method of the present invention responds to the above-described needs. In particular, the method of the present invention is based on a metagenomic analysis of the entire intestinal microbiota of an individual, i.e. the typing of the intestinal microbiota. In particular, the method is based on the use of massive sequencing techniques and comprises the determination of several significant indices of the state of health of the intestinal microbiota and consequently of the state of health of the individual to whom the intestinal microbiota belongs.

Based on the state of health of an individual, as determined by applying the method of the invention, it is possible to adopt manoeuvres to correct the lifestyle of the individual concerned, in particular by intervening in the diet and/or with an ad hoc, or personalised, pharmacological therapy, serving to improve the individual's state of health by normalising (balancing) the intestinal microbiota. Therefore, by applying the method of the invention it is possible to improve the general state of public health and, consequently, to considerably reduce the health care costs that every state is obliged to bear.

It should be noted that the characterisation of intestinal microbiota by means of massive sequencing techniques entails an appropriate bioinformatics analysis of the data in order to reconstruct the qualitative (microorganisms present), quantitative (proportion among the different microorganisms) and functional (impact on the host's health) composition of the entire intestinal microbiota (microbial ecosystem). In particular, this phase requires a high level of bioinformatics competence, as well as thorough knowledge of the ecosystem and the literature related to it. Through the method of the invention, one can advantageously characterise the intestinal microbiota of an individual by translating scientific data that is difficult to comprehend into an easily exploitable outcome relating to the composition and function of the microbiota in terms of deviation from a healthy composition (dysbiosis). In other words, the method of the present invention enables the state of health of an individual to be indirectly evaluated by measuring indices that translate the data related to the massive typing of the intestinal microbiota, which would otherwise difficult to comprehend and unusable for an individual, into more useful concepts such as prevention or risk in relation to various pathological conditions.

### DESCRIPTION OF THE FIGURES

The present invention will be described in detail below and also exemplified, by way of non-limiting illustration, with the aid of the following figures, in which:

FIG. 1 shows an example of a plot which represents the structure of an individual's intestinal microbiota resulting from the descriptive analysis of a faecal sample isolated from the individual, carried out with the method of the invention.

FIG. 2 shows an example of a typical table expressing the results of an analysis of the metabolic efficiency of the microbiota of a faecal sample measured with the method of the invention.

FIG. 3 shows an example of indices of association between the profile of the microbiota of a faecal sample measured with the method of the invention and the main disorders taken into consideration, in particular immunomodulation (A), inflammatory intestinal diseases (B), intestinal permeability (C), obesity-type 2 diabetes-metabolic syndrome (D) and aging (E).

### DETAILED DESCRIPTION OF THE INVENTION

A first general aspect of the present invention relates to a method for determining and/or monitoring the state of health and/or the balance of the intestinal microbiota of an individual, and therefore, indirectly, also the state of health of the individual to whom the microbiota belongs, comprising the steps of:
obtaining an isolated biological sample from an individual, preferably a faecal sample or a biopsy of an intestinal tract;
isolating, or purifying, the bacterial DNA from said sample;
typing the bacterial populations of said sample, where the set of said bacterial populations are likenable to (or representative of) the intestinal microbiota of said individual;
assigning the typed bacterial populations to at least one taxonomic unit selected from among: phylum, class, order, family, genus and species;
measuring the diversity index of said intestinal microbiota compared to a reference index represented by the intestinal microbiota of a population of healthy individuals;
measuring the index of anti-inflammatory and/or immunomodulating potential of said intestinal microbiota compared to a reference index represented by the intestinal microbiota of a population of healthy individuals;
measuring the index of association between said intestinal microbiota and at least one pathological condition compared to the reference index calculated considering the intestinal microbiota of a population of healthy individuals;
measuring the index of association between said intestinal microbiota and systemic aging compared to the reference index calculated considering the intestinal microbiota of a population of healthy individuals.

In this context, the term “intestinal microbiota” identifies the bacterial populations (i.e. the billions of bacteria) which colonise the gastrointestinal tract of each individual.

In this context, “determining the state of health of an individual” means evaluating the profile of the intestinal microbiota (relative abundance of one or more taxonomic units present inside the gastrointestinal tract) and consequently evaluating the involvement of said microbiota in preventing and/or favouring the onset and/or the establishment of local and/or systemic disorders and/or pathological conditions. The determination/evaluation of an individual's state of health with the method of the present invention is thus indirect.

According to a preferred aspect, the faecal sample can be taken by the individual autonomously, preferably using a spatula or a brush, and introducing the sample into a special container.

Before carrying out the step of isolating the bacterial DNA it is preferable to homogenise the sample, preferably using a homogeniser, for example a Stomacher.

An aliquot of the sample is subjected to the step of bacterial DNA extraction.

It is preferable to use an amount of sample such as to obtain a sufficient amount of bacterial DNA to be subjected to the subsequent amplification step, i.e. not below the detection (sensitivity) limit of the method used for amplification. More preferably, the amount of sample used for the extraction step ranges from 100 to 500 mg, more preferably from 200 to 400 mg, even more preferably it is about 250 mg.

The DNA extraction step is carried out with the normal techniques known to the person skilled in the art for such a purpose, for example, by using kits, or with the classic methods that use phenol-chloroform. The extraction technique that is preferred for the purposes of the present invention is based on cell disruption by mechanical shaking in a lysis solution containing salts and protein denaturants. Preferably, the extraction is carried out in the presence of glass and/or zirconium beads. The sample thus treated is then brought to high temperatures, preferably to about 95° C. Then follows a step of removing the debris, preferably by centrifugation, and a step of removing the protein fraction, preferably using an ammonium acetate solution. The sample thus treated is preferably incubated at low temperatures and then subjected to centrifugation. The DNA in the solution is then separated, preferably by precipitation with alcohol, preferably isopropanol, and centrifugation.

Subsequently, the DNA is diluted, preferably in a buffer solution which preferably contains chelating agents.

In order to remove RNA and proteins it is advisable to treat the DNA with enzymes suited to this purpose.

Before proceeding with the typing of the bacterial DNA it is preferable to check the quality and/or quantity of the extracted bacterial DNA.

In order to carry out a qualitative check on the extracted bacterial DNA, it is possible to use any technique known to the person skilled in the art for such a purpose, for example DNA analysis by gel electrophoresis, or measuring the DNA/protein ratio, i.e. 260/280 nm.

In order to carry out a quantitative check on the extracted bacterial DNA, it is possible to use any technique known to the person skilled in the art for this purpose. Preferably, the concentration of bacterial DNA is checked by means of spectrophotometric and/or fluorimetric readings, for example using instruments such as, for example, NanoDrop or Qubit.

The step of typing (or identifying) the bacterial populations present in the sample is preferably carried out by amplifying at least one portion of the rRNA 16S gene by PCR (Polymerase Chain Reaction) and subsequent sequencing of the amplified DNA.

PCR is preferably carried out using at least one pair of primers which map in a conserved area of the gene and amplify a specific hypervariable region of the bacterial category (i.e. this sequence is species specific). In other words, by amplifying this region of the gene, sequencing it and comparing the sequence thereof with those present in the database, it will be possible to identify the bacterial populations present in the sample.

The primer pair that is preferred for the purposes of the present invention is SEQ ID NO: 1 (5′-CCTACGGGNGGCWGCAG-3′) and SEQ ID NO: 2 (5′-GACTACHVGGGTATCTAATCC-3) or sequences having 80-95% identicalness. In any case, other primer pairs can also be used.

Preferably, amplification by PCR comprises the denaturation of the DNA, which lasts about 3 minutes at about 95° C., and various cycles, preferably at least 25 cycles, of denaturation-annealing-elongation, each cycle comprising a denaturation step of about 30 seconds at about 95° C., then an annealing step of about 30 seconds at about 55° C. (the temperature of this step depends on the annealing temperature of the various primers used for amplification) and then an elongation step of about 30 seconds at about 72° C. The amplification ends with a final step of about 5 minutes at about 72° C.

The sequencing of the amplified DNA is carried out with any technique known to the person skilled in the art for such purposes. Particularly preferred for the purposes of the present invention is the method of sequencing on an Ion Torrent or Illumina MiSeq or MinION platform, following the specific protocols for each technique.

The set of bacterial populations identified in the sample can be likened to the intestinal microbiota of the individual to whom the sample belongs. Therefore, the technical characteristics shown by the set of the bacterial populations of the sample can be likened to those of the intestinal microbiota of the individual concerned.

The taxonomic assignment of the bacterial populations identified in the sample is preferably carried out using known algorithms to this end.

For the purposes of taxonomic assignment it is preferable to use Quantitative Insights Into Microbial Ecology (QIIME), which is a free bioinformatics pipeline. In this manner it will be possible to assign the bacterial populations identified to each taxonomic unit (taxonomic category), or phylum, class, order, family, genus or species.

In a preferred embodiment, the method also comprises a step of quantifying the bacterial populations for each taxonomic unit.

At this point, the method of the present invention comprises a step of descriptive analysis. This step comprises measuring (determining) the diversity index of the intestinal microbiota, i.e. the set of bacterial populations typed on the basis of the sample. Said diversity index is preferably calculated as the number of genera and/or families of bacterial populations typed in said sample under examination compared to the number of genera and/or families of bacterial populations typed in a control sample of healthy individuals. More specifically, this index is preferably calculated by placing the genera and/or families of bacterial populations typed in the sample under examination in descending order according to their relative abundance and adding together, in this order, the relative abundance of each until arriving at or exceeding 90%. The number of genera and/or families added together until reaching or exceeding 90% represents the value of the diversity index of the sample under examination.

This index depends, in particular, on the population taken as reference. Preferably, it ranges from 8 to 22, more preferably from 10 to 17. The diversity index is considered normal if it falls between 8 and 22, preferably between 10 and 17. Beyond these intervals, the diversity index is to be considered outside the normal range. When values of the index outside the normal range are determined, it means that the diversity of the intestinal microbiota measured in the sample under examination is greater than the maximum value or less than the minimum value of diversity in the control population of healthy individuals and this can lead to an imbalance in the functionality of the microbiota, with a consequent impact on the health of the individual to whom the microbiota considered belongs.

According to a preferred aspect of the method, the descriptive step comprises a further step of measuring relative abundances at the level of at least one taxonomic unit, preferably at the family level, i.e. the percentage of typed bacterial populations distributed as a taxonomic unit, preferably as a family, relative to the total bacterial populations typed with the method described here, preferably through the amplification and sequencing of a region of the rRNA 16S gene.

Preferably, the individual relative abundances can be calculated in comparison to a population of healthy individuals in the form of medians and/or percentiles (for example, 25th, 75th, 10th and 90th).

The taxonomic units, preferably bacterial families, analysed in the method described are preferably those which contribute significantly to the balance of the intestinal microbiota and, consequently, to the individual's state of health.

Particularly preferred are bacterial families selected from among: Bacillaceae, Enterococcaceae, Eubacteriaceae, Fusobacteriaceae, Lactobacillaceae, Leuconostocaceae, Methanobacteriaceae, Oxalobacteriaceae, Peptococcaceae, Prevotellaceae, Desulfovibrionaceae, Enterobacteriaceae, Peptostreptococcaceae, Porphyromonadaceae, Rikenellaceae, Clostridiaceae, Streptococcaceae, Verrucomicrobiaceae, Coriobacteriaceae, Erysipelotrichaceae, Veillonellaceae, Bifidobacteriaceae, Bacteroidaceae, Lachnospiraceae, and Ruminococcaceae.

The values of the relative abundances of the bacterial population of the sample and/or of the individual families and/or genera which fall outside the interval between the 10th and 90th percentiles of the reference population are considered values outside the normal range. When values outside the normal range are determined, it means that, for that particular taxonomic unit, preferably for that bacterial family, the relative abundance is too high or too low and this can lead to an imbalance in the functionality of the microbiota, with an impact on the health of the individual host.

The remaining values, i.e. those in the interval between the 10th and 90th percentiles considered, are considered normal values.

Preferably, the relative abundance at the level of at least one taxonomic unit, more preferably at the genus level, is shown as a percentage value.

Preferably, the bacterial genera for which relative abundances greater than or equal to 0.1% are measured will be considered relevant, i.e. greater than or equal to the detection (detectability) limit given 1000 evaluated sequences.

In a preferred embodiment, the method of the present invention further comprises a step of measuring the microbial dysbiosis index.

In the context of the present invention, “microbial dysbiosis index” means the degree of dysbiosis of the intestinal microbiota, i.e. the deviation of the intestinal microbiota of the individual considered from the balanced profile typical of the intestinal microbiota of a healthy adult individual.

This index is calculated as the ratio between the sum of the relative abundances of bacterial families and/or genera associated/associable with pathological conditions of a metabolic and/or inflammatory and/or immunological type and the sum of the relative abundances of bacterial families and/or genera associated/associable with the state of health of an individual (so-called health-promoting bacteria).

Preferably, the bacterial families and/or genera associated/associable with pathological conditions are selected from among: Bilophila, Desulfovibrio, Enterobacteriaceae, Fusobacterium, Erysipelotrichaceae, Campylobacter, Alistipes, Collinsella and Clostridium.

Preferably, the bacterial families and/or genera associated/associable with the state of health of an individual are selected from among: Bifidobacterium, Faecalibacterium, Roseburia, Coprococcus, Akkermansia, Lactobacillus, Lachnospiraceae, Christensenellaceae and Ruminococcaceae.

The microbial dysbiosis index is considered normal when it is below the maximum value calculated in the control population of healthy individuals. Above this value, the microbial dysbiosis index is to be considered outside the normal range. When values outside the normal range are determined, it means that the intestinal microbiota of the individual considered deviates significantly from the typical balanced profile of the intestinal microbiota of a healthy adult individual, and this can lead to an imbalance in the functionality of the microbiota, with an impact on the health of the individual host.

After the descriptive step, the method of the present invention comprises a step of measuring (determining) further significant indices of the state of health of the intestinal microbiota of an individual and thus of the state of health thereof.

In particular, the method comprises a step of measuring the index of anti-inflammatory and/or immunomodulating potential of the intestinal microbiota, i.e. of the bacterial population typed on the basis of the sample under examination, where said index ranges from 0 to 10 and is calculated on the basis of the relative abundance of at least one, preferably four, bacterial genera/families with an anti-inflammatory and/or immunomodulating potential relative to a healthy reference population.

In this context, the index of anti-inflammatory and/or immunomodulating potential measures the ability of the intestinal microbiota to interact with the immune system, favouring the correct functioning thereof, and thus promoting an individual's state of health. The value of the index ranges from 0 to 10. It has a value of zero when the intestinal microbiota of the individual is associated/associable with poor anti-inflammatory and/or immunomodulating activity—in other words, when all of the bacterial genera/families considered have a relative abundance below the 10th percentile calculated in the healthy reference population. The index has a value of 10 when the intestinal microbiota of the individual is associated/associable with excellent anti-inflammatory and/or immunomodulating activity—in other words, when all the bacterial genera/families considered have a relative abundance above the 25th percentile calculated in the healthy reference population.

According to a preferred aspect, the index of anti-inflammatory and/or immunomodulating potential is calculated by evaluating the relative abundance of at least one, preferably more than one, more preferably at least four, bacterial genera/families with an anti-inflammatory and/or immunomodulating potential compared to the relative abundance of the same bacterial genera/families in a healthy reference population. The contribution of each bacterial genus/family to the index is calculated on the basis of the weight or sum of weights assigned to said at least one bacterial genus/family.

Preferably, said weight ranges from 0 to 1. In particular, the weight corresponds to the frequency with which said bacterial genus/family is above the 10th percentile in a population of healthy individuals. Preferably, when the value of the relative abundance of said bacterial genus/family falls below the 10th percentile of the reference population, the weight of that genus/family is not included in the calculation of the index of anti-inflammatory and/or immunomodulating potential; when the relative abundance falls between the 10th and 25th percentiles of the reference population, a third of the weight calculated for said bacterial genus/family is counted; when the relative abundance falls above the 25th percentile of the reference population, the whole weight calculated for said bacterial genus/family is counted.

The index of anti-inflammatory and/or immunomodulating potential is preferably considered normal if the values of the index are comprised between 2.5 and 10. When the index is below 2.5 it is considered outside the normal range. When values outside the normal range are determined, it means that the intestinal microbiota of the individual considered has a poor anti-inflammatory and/or immunomodulating activity compared to the intestinal microbiota of a healthy adult individual, and this can lead to an imbalance in the functionality of the microbiota, with an impact on the health of the individual host.

Said at least one bacterial genus/family is preferably selected from among Faecalibacterium, Bifidobacterium, Akkermansia, Coprococcus, Lachnospira, Christensenellaceae and Roseburia; more preferably Faecalibacterium, Bifidobacterium, Akkermansia and Roseburia.

A further step of the method comprises measuring (determining) the index of association between the intestinal microbiota of said individual and at least one disorder and/or pathological condition with metabolic and/or immunological and/or inflammatory involvement, i.e. the index of intestinal microbiota involvement in favouring the onset and/or establishment of one or more disorders and/or pathological conditions with metabolic and/or immunological and/or inflammatory involvement.

In the context of the present invention, “disorder and/or pathological condition with metabolic and/or immunological and/or inflammatory involvement” means an alteration in human energy metabolism and/or in the correct functioning of the immune system and/or in the inflammatory state (local, at the level of the intestine, and/or systemic), which results in a clinical manifestation. Said clinical manifestation is preferably: obesity, type 2 diabetes, metabolic syndrome, non-alcoholic hepatic steatosis, insulin resistance, hypercholesterolaemia, deregulation of glucose metabolism, cardiovascular diseases, hypertension, Crohn's disease, ulcerative colitis, diverticular diseases, irritable bowel syndrome, allergies, food intolerances, diarrhoea, constipation, colitis and enteritis.

The index of association between the intestinal microbiota of said individual and at least one disorder and/or pathological condition with metabolic and/or immunological and/or inflammatory involvement preferably ranges from 0 to 10. In particular, it will have a value of zero when said intestinal microbiota is not associated/associable with (i.e. does not predispose to) said at least one disorder and/or pathological condition with metabolic and/or immunological and/or inflammatory involvement, whereas it will have a value of 10 when said intestinal microbiota is associated/associable with (i.e. predisposes to) said disorder and/or pathological condition with metabolic and/or immunological and/or inflammatory involvement.

The index of association between the intestinal microbiota of said individual and a disorder and/or pathological condition with metabolic and/or immunological and/or inflammatory involvement is preferably calculated on the basis of the weight or sum of the weights assigned to at least one bacterial genus/family associated/associable with said disorder and/or pathological condition with metabolic and/or immunological and/or inflammatory involvement having a relative abundance above the 90th percentile calculated for the corresponding distribution in a reference population of healthy individuals, and/or to at least one bacterial genus/family not associated/associable with said disorder and/or pathological condition with metabolic and/or immunological and/or inflammatory involvement having a relative abundance below the 10th percentile calculated for the corresponding distribution in a reference population of healthy individuals.

Preferably, said weight ranges from 0 to 1. In particular, in the case of the at least one bacterial genus/family associated/associable with said disorder and/or pathological condition with metabolic and/or immunological and/or inflammatory involvement, the weight corresponds to the frequency with which the relative abundance of said bacterial genus/family results in a population of individuals affected by said disorder and/or pathological condition with metabolic and/or immunological and/or inflammatory involvement above the 90th percentile of a reference population of healthy individuals; whereas, in the case of the at least one bacterial genus/family not associated/associable with said disorder and/or pathological condition with metabolic and/or immunological and/or inflammatory involvement, the weight corresponds to the frequency with which the relative abundance of said bacterial genus/family results in a population of individuals affected by said disorder and/or pathological condition with metabolic and/or immunological and/or inflammatory involvement below the 10th percentile of a reference population of healthy individuals.

Preferably, the bacterial genus/family associated with said disorder and/or pathological condition with metabolic and/or immunological and/or inflammatory involvement is selected from among: Erysipelotrichaceae, Ruminococcus:Lachnospiraceae, Prevotella, Enterobacteriaceae, Lactobacillus, Alistipes, Collinsella Desulfovibrionaceae, Campylobacter, Fusobacteriaceae and Megamonas.

Preferably, the bacterial genus/family not associated with said disorder and/or pathological condition with metabolic and/or immunological and/or inflammatory involvement is selected from among: Faecalibacterium, Akkermansia, Bifidobacterium, Roseburia and Christensenellaceae.

Preferably, the index of association between the intestinal microbiota of said individual and a disorder and/or pathological condition with metabolic and/or immunological and/or inflammatory involvement is considered normal if it has values comprised between the values 0 and 4.5.

Preferably, said index is considered outside the normal range if it has values above about 4.5. When values outside the normal range are determined, it means that the intestinal microbiota of the individual considered moderately (for values comprised between 4.5 and 6.5) or greatly (for values comprised between 6.5 and 10) predisposes to a disorder and/or pathological condition with metabolic and/or immunological and/or inflammatory involvement.

A further step of the method comprises measuring (determining) the index of association between said intestinal microbiota and systemic aging, i.e. the index of intestinal microbiota involvement in favouring disorders associated with aging.

In this context, “disorders associated with aging” means the progressive decline in immune system functionality (called immunosenescence) and the consequent greater susceptibility to infectious pathologies, as well as the chronic, generalised inflammatory state typically associated with immunosenescence, called inflammaging.

The index of association between said intestinal microbiota and the disorders associated with aging preferably ranges from 0 to 10. In particular, it has a value of zero when said intestinal microbiota is not associated/associable with disorders associated with aging, whereas it has a value of 10 when said intestinal microbiota greatly predisposes to disorders associated with aging.

The index of association between the intestinal microbiota and disorders associated with aging is preferably calculated on the basis of the weight or sum of weights assigned to at least one pro-aging bacterial genus/family having a relative abundance above the 90th percentile calculated for the corresponding distribution in a reference population of healthy adult individuals and/or at least one anti-aging bacterial genus/family having a relative abundance below the 10th percentile calculated for the corresponding distribution in a reference population of healthy adult individuals.

Preferably, said weight ranges from 0 to 1. In particular, in the case of the at least one pro-aging bacterial genus/family, the weight corresponds to the frequency with which the relative abundance of said bacterial genus/family results in a population of elderly individuals above the 90th percentile of a reference population of healthy adult individuals, i.e. of an age preferably comprised between 18 and 59 years; whereas, in the case of the at least one anti-aging bacterial genus/family, the weight corresponds to the frequency with which the relative abundance of said bacterial genus/family results in a population of elderly individuals below the 10th percentile of a reference population of healthy adult individuals. In this context, “elderly” individual means an individual aged 65 or older. Preferably, said pro-aging bacterial genus/family is selected from among: Enterobacteriaceae, Porphyromonadaceae, Rikenellaceae, Oscillospira, Desulfovibrionaceae, Unclassified_Clostridiales, Synergistaceae and Anaerotruncus. Preferably, said anti-aging genus/family is selected from among: Faecalibacterium, Bifidobacterium, Roseburia, Coprococcus and Akkermansia.

Preferably, the index of association between the intestinal microbiota and disorders associated with aging is considered normal if it has values comprised between 0 and 6. Preferably, said index is considered outside the normal range if it has values above about 6. When values outside the normal range are determined, it means that the intestinal microbiota of the individual considered moderately (for values comprised between 6 and 8) or greatly (for values comprised between 8 and 10) predisposes to disorders associated with aging.

In a further embodiment, the method comprises a further step of measuring an index associated with intestinal permeability, i.e. an index whose value is representative of the intestinal microbiota involvement in favouring (promoting) intestinal permeability, otherwise known as “leaky gut syndrome”.

In this context, “intestinal permeability” means an alteration in the correct functionality of the intestinal epithelium, as a result of which molecules and/or bacteria present in the intestinal lumen can pass through the intestinal wall and end up in the surrounding tissues, causing infections and inflammation.

The index of association between said intestinal microbiota and intestinal permeability preferably ranges from 0 to 10. In particular, it has a value of zero when said intestinal microbiota is not associated/associable with (i.e. does not favour) intestinal permeability, whereas it has a value of 10 when said intestinal microbiota is associated/associable with (i.e. favours) intestinal permeability.

The index of association between said intestinal microbiota and intestinal permeability is preferably calculated on the basis of the weight and/or of the sum of the weights assigned to at least one bacterial genus/family favouring intestinal permeability (predisposing variable), having a relative abundance above the 90th percentile calculated for the corresponding distribution in a reference population of healthy individuals, and/or the potential production of butyrate (protective variable), having values below the 10th percentile calculated for the corresponding distribution in a reference population of healthy individuals.

Preferably, said weight ranges from 0 to 1. In particular, in the case of the at least one bacterial genus/family favouring intestinal permeability, the weight corresponds to the frequency with which the relative abundance of said bacterial genus/family results in a population of individuals affected by leaky gut syndrome above the 90th percentile of a reference population of healthy individuals; whereas, in the case of the potential production of butyrate, the weight corresponds to the frequency with which said potential results in a population of individuals affected by leaky gut syndrome below the 10th percentile of a reference population of healthy individuals. Preferably, said bacterial genus/family favouring intestinal permeability is selected between Desulfovibrionaceae and Enterobacteriaceae (predisposing variable). Preferably, said potential production of butyrate by the microbiota considered is calculated as the sum of the relative abundances of at least one between bacterial genera or families known to produce butyrate, preferably selected from among: Faecalibacterium, Roseburia, Anaerostipes, Coprococcus, Porphyromonas, Shuttleworthia, Butyrivibrio, Odoribacter and Megasphaera. Preferably, the index of association between said intestinal microbiota and intestinal permeability is considered normal if it has values comprised between the values 0 and 7. Preferably, said index is considered outside the normal range if it has values above about 7. When values outside the normal range are determined, it means that the intestinal microbiota of the individual considered greatly predisposes to intestinal permeability.

In a further embodiment, the method comprises a further step of evaluating (determining) the metabolic efficiency of the intestinal microbiota of the individual considered.

Said step comprises determining at least one of the following activities of the intestinal microbiota considered: production of acetate, production of butyrate, production of propionate, production of lactate, production of hydrogen sulphide, production of bacterial LPS (lipopolysaccharide), mucolysis or proteolysis.

In order to determine the activity of producing acetate, the method preferably comprises measuring the relative abundance in the sample under examination, compared to a reference population of healthy individuals, of at least one bacterial genus/family selected from among: Bifidobacterium, Blautia, Ruminococcus:Ruminococcaceae, Bacteroides, Odoribacter and Alistipes.

The value obtained is compared to the value obtained by means of the same calculation in a healthy reference population. It is considered normal if it falls in the interval between the 10th and 90th percentiles of the reference population of healthy individuals.

In order to determine the activity of producing butyrate, the method comprises measuring the relative abundance in the sample under examination, compared to a reference population of healthy individuals, of at least one bacterial genus/family preferably selected from among: Faecalibacterium, Roseburia, Anaerostipes, Coprococcus, Porphyromonas, Shuttleworthia and Butyrivibrio, Odoribacter and Megasphaera.

The value obtained is compared to the value obtained by means of the same calculation in a healthy reference population. It is considered normal if it falls in the interval between the 10th and 90th percentiles of the population of healthy individuals of reference.

In order to determine the activity of producing propionate, the method comprises measuring the relative abundance in the sample under examination, compared to a reference population of healthy individuals, of at least one bacterial genus/family preferably selected from among: Bacteroides, Parabacteroides, Veillonellaceae, Coprococcus, Roseburia, Ruminococcus:Lachnospiraceae and Odoribacter.

The value obtained is compared to the value obtained by means of the same calculation in a healthy reference population. It is considered normal if it falls in the interval between the 10th and 90th percentiles of the population of healthy individuals of reference.

In order to determine the activity of producing lactate, the method comprises measuring the relative abundance in the sample under examination, compared to a reference population of healthy individuals, of at least one bacterial genus/family preferably selected from among: Lactobacillus, Bifidobacterium, Roseburia and Faecalibacterium.

The value obtained is compared to the value obtained by means of the same calculation in a healthy reference population. It is considered normal if it falls in the interval between the 10th and 90th percentiles of the population of healthy individuals of reference.

In order to determine the activity of producing hydrogen sulphide, the method comprises measuring the relative abundance in the sample under examination, compared to a reference population of healthy individuals, of at least one bacterial genus/family preferably selected from among: Bilophila, Desulfovibrio and other genera belonging to the family Desulfovibrionaceae.

The value obtained is compared to the value obtained by means of the same calculation in a healthy reference population. It is considered normal if it falls in the interval between the 10th and 90th percentiles of the population of healthy individuals of reference.

In order to determine the activity of producing LPS, the method comprises measuring the relative abundance in the sample under examination, compared to a reference population of healthy individuals, of at least one bacterial genus/family preferably selected from among: Enterobacteriaceae, Fusobacterium and Bacteroides.

The value obtained is compared to the value obtained by means of the same calculation in a healthy reference population. It is considered normal if it falls in the interval between the 10th and 90th percentiles of the population of healthy individuals of reference.

In order to determine the activity of mucolysis, the method comprises measuring the relative abundance in the sample under examination, compared to a reference population of healthy individuals, of at least one bacterial genus/family preferably selected from among: Akkermansia, Bacteroides, Ruminococcus:Lachnospiraceae.

The value obtained is compared to the value obtained by means of the same calculation in a healthy reference population. It is considered normal if it falls in the interval between the 10th and 90th percentiles of the population of healthy individuals of reference.

In order to determine the activity of proteolysis, the method comprises measuring the relative abundance in the sample under examination, compared to a reference population of healthy individuals, of at least one bacterial genus/family preferably the Clostridium.

The value obtained is compared to the value obtained by means of the same calculation in a healthy reference population. It is considered normal if it falls in the interval between the 10th and 90th percentiles of the population of healthy individuals of reference.

In one embodiment, the method comprises a further step of identifying, in the typed bacterial population, at least one potentially pathogenic bacterial subpopulation.

In this context, “potentially pathogenic bacteria” means a bacterial species/genus that can be detected in the microbiota of a healthy individual in a very low abundance but which can give rise to clinically significant manifestations (e.g. diarrhoea) if the relative abundance thereof increases.

Preferably, said at least one potentially pathogenic bacterial population belongs to a species selected from among: Bacteroides fragilis, Enterococcus faecalis, Clostridium perfringens, Clostridium difficile, Bacillus cereus, Helicobacter pylori and Pseudomonas aeruginosa and/or the genera Klebsiella, Salmonella, Campylobacter, Yersinia, Proteus, Vibrio, Aeromonas and Listeria.

Preferably, in the method of the invention, said bacteria are deemed to be present when they exceed the detection limit of at least one OTU (Operational Taxonomic Unit), identified as belonging to the species/genus of interest, which has been assigned at least two of the sequences obtained by DNA sequencing and analysis with bioinformatics algorithms, preferably the bioinformatics pipeline QIIME.

According to a preferred embodiment, for the purpose of defining the balance of the intestinal microbiota examined, an overall evaluation is made of the indices of the descriptive analysis (diversity index and microbial dysbiosis index), the anti-inflammatory and immunomodulating potential, and the indices of microbiota involvement in favouring intestinal permeability, systemic aging, and the onset or establishment of inflammatory intestinal diseases, pathological conditions with metabolic involvement and colorectal cancer.

According to the method of the invention, intestinal microbiota is defined dysbiotic, i.e. the state of health of the individual is altered, preferably when it exhibits at least one of the following conditions:

1) diversity index and/or microbial dysbiosis index outside the normal range compared to the control population of healthy individuals; and/or

2) anti-inflammatory and immunomodulating potential below a value of 2.5, indicative of a poor anti-inflammatory and immunomodulating activity of the intestinal microbiota; and/or

3) at least one of the following indices above the specified threshold value: index of microbiota involvement in favouring intestinal permeability above 7; index of microbiota involvement in favouring systemic aging above 6; index of microbiota involvement in favouring the onset or establishment of inflammatory intestinal diseases above 4.5; index of microbiota involvement in favouring pathological conditions with metabolic involvement above 4.5; index of microbiota involvement in favouring colorectal cancer above 7.

When the method of the invention is applied, if the microbiota proves to be dysbiotic, it is preferable also to analyse the maintenance (or lack thereof) of ecosystem functionality, i.e. the production of butyrate, acetate, propionate, lactate and hydrogen sulphide, mucolytic activity or proteolytic activity as previously described, and preferably also consider the relative abundances of the principal bacterial genera/families making up the ecosystem, with the aim of identifying the bacterial groups that are overabundant and those that are underrepresented, i.e. the ones that fall outside the interval between the 10th and 90th percentiles of the control population of healthy individuals.

These data make it possible to derive information on the balance or dysbiosis of the intestinal microbiota analysed, the possible causes of the dysbiosis, preferably also considering the data of the available literature and any information that may have been provided by the individual to whom the intestinal microbiota belongs, and to evaluate the possible effect on the physical wellbeing of said individual.

Based on the identification of bacterial groups that are overabundant and/or underrepresented in the intestinal microbiota analysed and the available literature which demonstrates an effect of a dietary component or supplement on certain bacterial groups of the intestinal microbiota, it is also possible to give suggestions for intervening in the microbiota, in terms of nutritional and/or therapeutic guidelines (the latter in the form of advice on how to use types of food supplements and/or supplements based on probiotics). For example, one might suggest an increased consumption and/or inclusion in the diet of foods (and/or supplements) for which the available literature reports a positive effect on the growth of intestinal bacterial groups that are underrepresented in the intestinal microbiota analysed; similarly, one might suggest avoiding and/or decreasing the consumption of foods (and/or supplements) which, according to the available literature, promote the growth of microorganisms that are already overabundant in the intestinal microbiota analysed, or hinder that of microorganisms that are underrepresented in the aforesaid microbiota.

A further aspect of the present invention relates to a kit for implementing the method of the present invention, which comprises:

(i) a container for collecting faeces, said container preferably containing a spatula, preferably suitable for collecting a useful amount of faeces;

(ii) sample collecting instructions;

(iii) questionnaire suitable for collecting personal data, information on the state of health and/or eating habits of the individual.

The sample is preferably subjected to the method of the invention within 24-48 hours after being collected.

### EXAMPLE

In this example, the method of the present invention comprises the steps of calculating a set of indices which measure the intestinal microbiota involvement in different aspects of an individual's state of health. Based on the values obtained for these indices, it is possible to evaluate/monitor the individual's state of health. The indices are calculated on the basis of the sequences of a portion of the rRNA 16S gene amplified from the total bacterial DNA extracted from a faecal sample of the individual. The set of bacterial populations present in the faecal sample of an individual can be likened to the intestinal microbiota.

Processing of the Sample

Each sample of faecal material is processed as described below:
homogenisation of the faecal sample;
weighing of about 250 mg of homogenised sample for extraction of the DNA;
extraction of the total microbial DNA from the sample of faeces following the protocol published by A. Salonen et al., in J Microbiol Methods 2010;
qualitative and quantitative determination of the total microbial DNA via NanoDrop and/or Qubit.
typing of the bacterial populations present in the faecal sample by:

1) PCR amplification of a portion of the rRNA 16S gene (i.e. the gene encoding the 16S ribosomal subunit) using the primer pair Bact-0341 (5′-CCTACGGGNGGCWGCAG-3′) and Bact-0785 (5′-GACTACHVGGGTATCTAATCC-3) (Klindworth A, Pruesse E, Schweer T, Peplies J, Quast C, Horn M, Glockner FO. Evaluation of general 16S ribosomal RNA gene PCR primers for classical and next-generation sequencing-based diversity studies. Nucleic Acids Res. 2013 Jan. 7; 41(1):e1);

2) sequencing of the PCR product via the Ion Torrent or Illumina MiSeq or MinION platform;

3) analysis of the sequences of step (2) with the free QIIME (Quantitative Insights Into Microbial Ecology) pipeline so as to obtain: the count of each OTU (Operational Taxonomic Unit) detected, the taxonomic assignment and relative abundances of the bacterial populations at the level of phylum, class, order, family, genus and species (Caporaso J G, Kuczynski J, Stombaugh J, Bittinger K, Bushman F D, Costello E K, Fierer N, et al. QIIME allows analysis of high-throughput community sequencing data. Nat Methods. 2010 May; 7(5):335-6).

Descriptive Analysis

A descriptive analysis of the information obtained by processing the sample is subsequently performed. In particular, the relative abundances of each bacterial population identified in the faecal sample at the level of family are measured. In other words, a calculation is made of the percentage of sequences assigned to each family relative to the total sequences analysed (and filtered) for the faecal sample under examination.

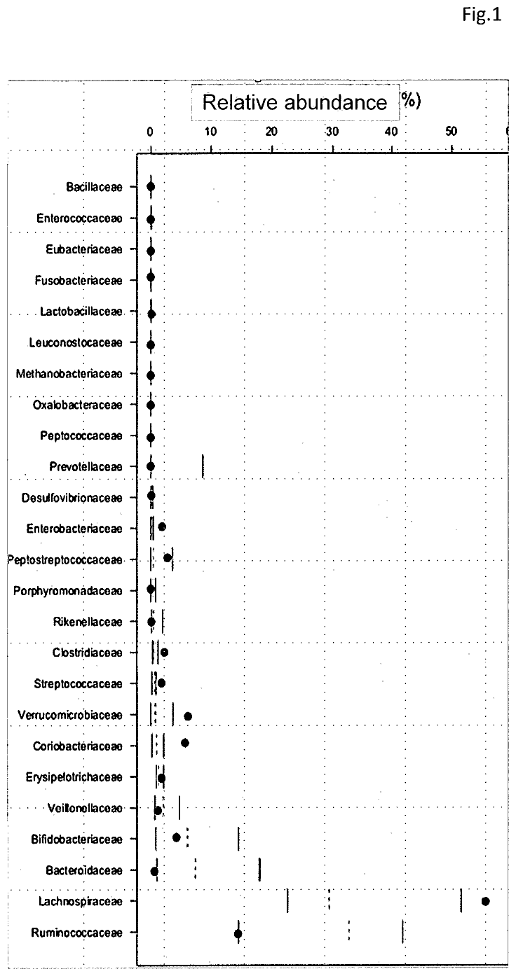

These data are combined in a single plot, an example of which is shown in FIG. 1. It shows the structure of the intestinal microbiota of an individual resulting from the descriptive analysis of the faecal sample of the individual considered. In particular, the individual relative abundances are shown compared to medians and percentiles (25th, 75th, 10th and 90th) obtained from a control sample made up of a population of healthy Italian individuals.

In this example, the bacterial families of greatest importance for the balance of the ecosystem and for their contribution to an individual's state of health were selected based on the available literature (reference families).

The reference bacterial families are: Bacillaceae, Enterococcaceae, Eubacteriaceae, Fusobacteriaceae, Lactobacillaceae, Leuconostocaceae, Methanobacteriaceae, Oxalobacteriaceae, Peptococcaceae, Prevotellaceae, Desulfovibrionaceae, Enterobacteriaceae, Peptostreptococcaceae, Porphyromonadaceae, Rikenellaceae, Clostridiaceae, Streptococcaceae, Verrucomicrobiaceae, Coriobacteriaceae, Erysipelotrichaceae, Veillonellaceae, Bifidobacteriaceae, Bacteroidaceae, Lachnospiraceae, and Ruminococcaceae.

In addition to the above-mentioned plot, one can also produce a plot of the relative abundance of each of the bacterial families analysed in order to have greater resolution in the comparison between the intestinal microbiota of the individual considered and the reference dataset (healthy control). In these plots, the values that fall outside the interval between the 10th and 90th percentiles of the reference dataset, i.e. values outside the normal range, are indicated with a reference colour (grey in the example). The other values, i.e. the normal ones, are indicated with a different colour (black in the example).

The method also provides data on the relative abundances at the level of bacterial genus, i.e. the percentage of sequences assigned to each bacterial genus relative to the total analysed sequences obtained for the faecal sample under examination.

In particular, these data are reported as a numerical percentage and only the genera with a relative abundance greater than or equal to 0.1% are provided.

The phylogenesis (family, order, class and phylum it belongs to) is given for each genus.

Finally, the descriptive analysis of the data related to the bacterial populations of the faecal sample under examination (i.e. the intestinal microbiota) is completed by the calculation of a diversity index and/or a dysbiosis index, whose value is presented in relation to the values of the same index calculated for a population of healthy individuals in the reference dataset.

The diversity index expresses the degree of biodiversity of the intestinal microbiota and is calculated on the basis of the number of bacterial genera/families detected by sequencing of the amplified DNA from the faecal sample.

The microbial dysbiosis index expresses the degree of dysbiosis, i.e. the deviation of the intestinal microbiota of the individual considered from the typical balanced profile of the intestinal microbiota of a healthy adult individual.

The calculation method measures the ratio between the sum of the relative abundances of the bacterial genera/families associated/associable with pathological conditions and the sum of the relative abundances of the bacterial genera/families associated/associable with a healthy status of an individual (so-called health-promoting bacteria).

In the example, the bacterial genera/families associated/associable with pathological conditions are the following: Bilophila, Desulfovibrio, Enterobacteriaceae, Fusobacterium, Erysipelotrichaceae, Campylobacter, Alistipes and Collinsella; whereas the bacterial genera/families associated/associable with a healthy status of an individual are the following: Bifidobacterium, Faecalibacterium, Roseburia and Akkermansia. For the purposes of the method of the present invention, the diversity index, which, as explained above, measures the heterogeneity of the bacterial populations (in terms of genus) of the faecal sample under examination and, therefore, of the intestinal microbiota of the individual to whom the faecal sample belongs, is the most relevant index for the purposes of the descriptive analysis of the information obtained by processing the sample.

Analysis of the Main Functional Classes

The method of the present invention further comprises giving results regarding the metabolic efficiency of the intestinal microbiota considered. In particular, metabolic efficiency is expressed at least as the cumulative capacity for the: production of acetate, production of butyrate, production of propionate, production of lactate, production of hydrogen sulphide, production of bacterial LPS (lipopolysaccharide) and mucolysis and proteolysis.

For each of these functions a sum is made of the relative abundance of bacterial families and/or genera selected on the basis of their ability to carry out the activity considered based on the data provided by the available literature.

Examples of families and/or genera selected for each functional class are given below:
For the production of acetate: Bifidobacterium, Blautia, Ruminococcus:Ruminococcaceae, Bacteroides, Odoribacter and Alistipes.
For the production of butyrate: Faecalibacterium, Roseburia, Anaerostipes, Coprococcus, Porphyromonas, Shuttleworthia, Butyrivibrio, Odoribacter and Megasphaera.
For the production of propionate: Bacteroides, Parabacteroides, Veillonellaceae, Coprococcus, Roseburia, Ruminococcus:Lachnospiraceae, and Odoribacter.
For the production of lactate: Lactobacillus, Bifidobacterium, Roseburia, and Faecalibacterium.
For the production of hydrogen sulphide: Bilophila and Desulfovibrio.
For the production of bacterial LPS: Enterobacteriaceae, Fusobacterium, Bacteroides.
For mucolysis: Akkermansia, Bacteroides, Ruminococcus:Lachnospiraceae.
For proteolysis: Clostridium.

The value obtained is compared with the values obtained with the same calculation performed for a control population made up of healthy individuals.

On the basis of the median and percentiles of the control dataset, a confidence interval is established, which goes from the 10th to the 90th percentile; outside this interval the specific metabolic function is associated with a circle in a reference colour (black in the example) which indicates, respectively, a “deficiency” or “excess” in that function.

Inside the confidence interval, the values falling between the 10th and the 25th percentiles and between the 75th and the 90th percentiles of the control dataset, are indicated with a circle in a reference colour (light grey in the example), which indicates a tendency toward a deficiency or excess, respectively, in the specific metabolic function.

Values that fall inside the interval between the 25th and 75th percentiles are assigned a circle in a reference colour (dark grey in the example), which indicates a “fully normal” metabolic function.

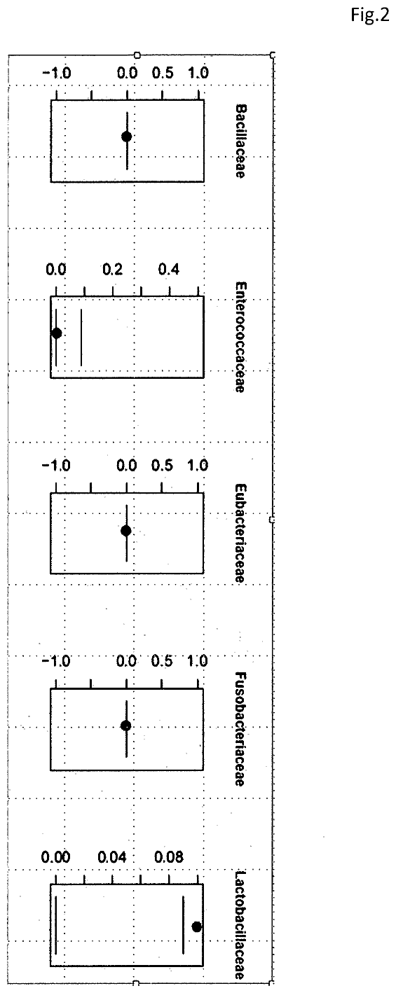

FIG. 2 shows a typical table expressing the results of the analysis of the metabolic efficiency of the microbiota in a faecal sample measured with the method of the present invention.

Detection of the Presence of Potentially Pathogenic Bacterial Groups

A further step of the method relates to the detection of sequences belonging to potentially pathogenic bacteria, for example those belonging to the species Bacteroides fragilis, Enterococcus faecalis, Clostridium perfringens, Clostridium difficile and the genera Klebsiella and Salmonella. Above the detection limit, the data are shown as “detected” (in grey) or “not detected”, since these are bacteria whose possible proliferation could result in episodes of clinical relevance.

Indices of the Health of the Intestinal Microbial Ecosystem

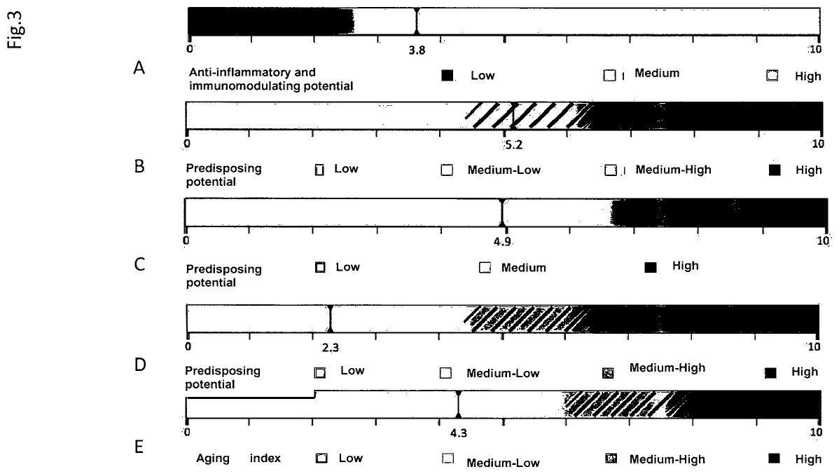

The method of the present invention is also based on the calculation of highly informative and relevant indices regarding the health of the intestinal microbiota. FIG. 3 shows an example of indices of association between the profile of the microbiota of an individual and the main disorders taken into consideration. Said indices are calculated on the basis of the data of relative abundance at the level of genus and/or family of bacteria identified in the faecal sample under examination.

Index of Anti-Inflammatory and Immunomodulating Potential

The index of anti-inflammatory and immunomodulating potential is expressed with a value ranging from 0 to 10. A value of the index equal to 0 is associated with a poor anti-inflammatory and immunomodulating activity of said intestinal microbiota; whereas a value of the index equal to is associated with an excellent anti-inflammatory and immunomodulating activity.

More specifically, in this example embodiment of the method, the index in question is calculated by selecting the four bacterial genera with the largest anti-inflammatory potential, i.e. the genera Faecalibacterium, Bifidobacterium, Akkermansia and Roseburia.

For each of them, a calculation is made of the relative abundance in the faecal sample under examination, compared to the corresponding distribution (percentiles and medians) in the reference dataset (healthy control), and a “weight” is attributed, expressed in a value between 0 and 1.

For each bacterial genus, when the relative abundance in the faecal sample under examination falls below the 10th percentile calculated for the reference dataset, the weight of that genus is not counted in the calculation of the index of anti-inflammatory and/or immunomodulating potential.

In order also to identify situations, tending to a low level of anti-inflammatory activity, when the relative abundance in the faecal sample under examination falls between the 10th and the 25th percentiles of the reference dataset, a third of the weight calculated for said bacterial genus is counted.

When the relative abundance in the faecal sample under examination falls above the 25th percentile of the reference dataset, the whole weight calculated for said bacterial genus/family is counted.

The value obtained after the necessary operations have been carried out represents the value of the index of anti-inflammatory and immunomodulating activity attributable to the microbiota belonging to the faecal sample analysed and thus to the individual to whom said sample belongs.

A microbiota with an excellent anti-inflammatory and immunomodulating activity shows an index with values between 5.5 and 10, represented in a reference colour (dark grey in the example); a microbiota with a medium anti-inflammatory and immunomodulating activity shows an index with values between 2.5 and 5.5, represented in another reference colour (light grey in the example); and a microbiota with poor anti-inflammatory and immunomodulating activity shows an index with values between 0 and 2.5, represented in a further reference colour (black in the example).

Index of Microbiota Involvement in Promoting Metabolic Disorders Such as Obesity, Type 2 Diabetes and Metabolic Syndrome

This index is expressed with a value from 0 to 10. A value of the index equal to 10 corresponds to (denotes) an intestinal microbiota which favours in the individual the onset or establishment of at least one of the following metabolic disorders: obesity, type 2 diabetes or metabolic syndrome. In other words, the intestinal microbiota of an individual for whom a value of the index equal to 10 is obtained will be associated/associable with at least one of said pathologies. A value of the index close to 0 is indicative of an intestinal microbiota capable of contributing to the individual's normal metabolic functions. That is, the intestinal microbiota of an individual for whom a value of the index close to 0 is obtained will not be associated/associable with said pathologies, because the intestinal microbiota is capable of fulfilling the individual's normal metabolic functions.

In this example, the index in question has been calculated using a “Bayesian case model” and a dataset of at least 40 obese subjects and/or subjects with type 2 diabetes (reference dataset).

In this case, the calculation of the index is based on the definition of obesogenic and anti-obesogenic bacterial genera/families and on the attribution of an “obesogenic weight” to each bacterial genus/family. The obesogenic bacterial genera/families are the following:

Erysipelotrichaceae, Ruminococcus:Lachnospiraceae, Prevotella, Enterobacteriaceae, Lactobacillus, Alistipes, Collinsella and Megamonas, whereas the anti-obesogenic bacterial genera/families are the following: Faecalibacterium, Akkermansia, Bifidobacterium and Roseburia The final obesogenic index expresses to what degree the intestinal microbiota of the individual considered is predisposed to favouring the onset or establishment of at least one of the following metabolic disorders: obesity, type 2 diabetes and metabolic syndrome, i.e. the association between the intestinal microbiota and said pathologies. The final obesogenic index is the result of a computation which considers the obesogenic and anti-obesogenic potential of each of the at least 12 (8+4) reference bacterial genera/families and the specific individual profile of relative abundance of these microorganisms in the faecal sample under examination compared to the healthy control.

Method of Calculating the “Obesogenic Weight” of the Reference Bacterial Groups:

Each obesogenic bacterial genus/family is assigned an “obesogenic weight” in terms of its contribution to the predisposition to obesity. The values of the obesogenic weight, corresponding to the actual bacterial obesogenic potential, is comprised between 0 and 1 and is equivalent to the frequency with which, in a dataset of at least 40 obese and/or diabetic subjects, the relative abundance of the bacterial genus/family is above the 90th percentile calculated for the corresponding distribution in the healthy control population.

Each anti-obesogenic bacterial genus/family is assigned an “anti-obesogenic weight” in termini of its contribution to the predisposition to obesity. The value of the obesogenic weight is comprised between 0 and 1 and corresponds to the obesogenic potential associated with the lack of the microorganism, equivalent to the frequency with which, in obese and/or diabetic subjects, the relative abundance of the bacterial genus/family is below the 10th percentile calculated for the corresponding distribution in the healthy control population.

Calculation of the Maximum “Obesogenic Weight” of the Intestinal Microbiota:

The sum of the above values obtained represents the maximum value that can be reached by the individual considered with intestinal microbiota that greatly predisposes to disorders of a metabolic type such as obesity, type 2 diabetes and metabolic syndrome.

In order to facilitate the graphic representation and reading of this index, a mathematical proportion has been used to distribute the values of the obesogenic index on a scale from 0 to 10, in which the maximum obesogenic weight is made equal to 10.

Calculation of the Individual Obesogenic Index:

The value of the obesogenic index of the intestinal microbiota for each individual considered is obtained by adding together the “obesogenic weights” of the bacterial genera/families (among the 12 mentioned above) which in the individual have a relative abundance above the 90th percentile of the corresponding distribution in healthy individuals, in the case of the obesogenic groups, or below the 10th percentile of the corresponding distribution in healthy individuals, in the case of the anti-obesogenic groups.

The value obtained is then compared against a scale of 0 to 10 and shown on the graph as a coloured bar to facilitate the reading thereof.

A normal index has values comprised between 0 and 2.5, represented in a reference colour (dark grey in the example); a microbiota that moderately predisposes to metabolic disorders shows an index with values between 2.5 and 4.5, represented in another reference colour (light grey in the example); a microbiota that predisposes to metabolic disorders shows an index with values between 4.5 and 6.5, represented in another reference colour (dark grey and crossed out in the example); and a microbiota that greatly predisposes to metabolic disorders shows an index with values between 6.5 and 10, represented in a further reference colour (black in the example).

Index of Microbiota Involvement in Favouring the Onset or Establishment of Inflammatory Intestinal Diseases

This index is expressed with a value of 0 to 10, where 10 corresponds to a highly inflammatory intestinal microbiota. In this example, the index is based on the definition of pro-inflammatory and anti-inflammatory bacterial genera/families and on the attribution of an “inflammatory weight” to each of them. In this example the following bacterial genera/families are defined as pro-inflammatory: Enterobacteriaceae, Desulfovibrionaceae and Campylobacter; and the following bacterial genera/families as anti-inflammatory: Faecalibacterium, Akkermansia, Bifidobacterium and Roseburia.

More specifically, the index in question is calculated using “Bayesian case model” and a dataset of at least 20 individuals with ulcerative colitis.

The final index expresses the degree to which the intestinal microbiota of the individual considered is predisposed to inducing at least one inflammatory pathology of the intestine, for example ulcerative colitis, Crohn's disease or diverticulitis.

Method of Calculating the “Inflammatory Weight” of the Reference Bacterial Groups:

The method is based on assigning each pro-inflammatory bacterial genus/family) an “inflammatory weight” in terms of its contribution to the predisposition to at least one inflammatory intestinal pathology. It is expressed in a value comprised between 0 and 1, corresponding to the actual inflammatory potential of the microorganism and equivalent to the frequency with which, in a dataset of at least 20 individuals with an inflamed intestine, the relative abundance of the bacterial genus/family is above the 90th percentile of the corresponding distribution in the healthy control population.

Each anti-inflammatory bacterial genus/family is also assigned an “inflammatory weight” in terms of its contribution to the predisposition to at least one inflammatory intestinal disease. It is expressed in a value comprised between 0 and 1, corresponding to the inflammatory potential associated with the lack of the microorganism and equivalent to the frequency with which, in subjects with an inflamed intestine, the relative abundance of the bacterial genus/family is below the 10th percentile of the corresponding distribution in the healthy control population.

Calculation of the Maximum “Inflammatory Weight” of the Intestinal Microbiota:

At this point a sum is made of the values of the index as obtained above, in particular, a sum of the values corresponding to the pro-inflammatory and anti-inflammatory bacterial genera/families. The sum represents the maximum value that can be reached by an individual with intestinal microbiota that greatly predispose to at least one inflammatory pathology.

In order to facilitate the graphic representation and reading of this index a mathematical proportion has been used to distribute the values of the index on a scale from 0 to 10, in which the maximum inflammatory weight is made equal to 10.

Calculation of the Individual Inflammatory Index:

The value of the inflammatory index of the intestinal microbiota of the individual considered has been calculated by adding together the “inflammatory weights” of the bacterial genera/families described above, which, in the individual, have a relative abundance above the 90th percentile of the corresponding distribution in healthy subjects, in the case of pro-inflammatory bacterial genera/families, or below the 10th percentile of the corresponding distribution in healthy subjects, in the case of anti-inflammatory bacterial genera/families.

The value obtained is then compared against the scale from 0 to 10 and shown on the graph as a coloured bar to facilitate the reading thereof.

A normal index shows values comprised between 0 and 2.5, represented in a reference colour (dark grey in the case considered); an index of an intestinal microbiota that moderately predisposes to at least one inflammatory intestinal disease shows values comprised between 2.5 and 4.5, shown in a further reference colour (light grey in the example); an index of intestinal microbiota that predisposes to at least one inflammatory intestinal disease shows values comprised between 4.5 and 6.5, shown in yet another reference colour (dark grey with stripes in the example); and an index of an intestinal microbiota that greatly predisposes to at least one inflammatory intestinal disease shows values comprised between 6.5 and 10, shown in a further reference colour (black in the example).

Index of Microbiota Involvement in Favouring the Onset or Establishment of Colorectal Cancer

This index is expressed with a value from 0 to 10. A value of 10 corresponds to (denotes) an intestinal microbiota that predisposes to colorectal cancer, i.e. an intestinal microbiota associated with colorectal cancer.

In this example, the index is based on the definition of:
predisposing variables, including 1) the relative abundance of the following bacterial genera/families: Desulfovibrionaceae, Coriobacteriaceae, Prevotellaceae, Fusobacterium, Campylobacteriaceae, Staphylococcaceae, Parvimonas; and 2) the detection of the presence of potentially pathogenic bacterial species, for example Bacteroides fragilis, Enterococcus faecalis; and
the detection of the presence of the following anti-tumour variables: relative abundance of the following bacterial genera/families: Faecalibacterium, Lactobacillus, Bifidobacterium, Roseburia and the potential production of butyrate.

A weight is attributed for each of these variables. The final index expresses the degree of intestinal microbiota involvement in favouring the onset and/or establishment of colorectal cancer and is the result of a computation that considers the pro- and anti-tumour potential of each at least of the aforesaid reference variables and the specific individual profile of relative abundance of the microorganisms involved.

As said earlier, each of the variables is assigned a “weight” expressed in a value comprised between 0 and 1.

For each individual considered, the value of the index of intestinal microbiota involvement in favouring the onset and/or establishment of colorectal cancer is obtained by adding together the “weights” of the aforesaid variables, which in the individual have, in the case of the predisposing variables, a value above the 90th percentile of the corresponding distribution in healthy individuals (or, in the case of potentially pathogenic species, the presence thereof has simply been detected), or, in the case of anti-tumour variables, a value below the 10th percentile of the corresponding distribution in healthy individuals.

In order to facilitate the reading thereof, the value obtained is then compared against the scale from 0 to 10 and shown on a coloured bar.

A normal index shows values comprised between 0 and 4; an index of a microbiota that moderately predisposes to colorectal cancer shows values comprised between 4 and 7; an index of a microbiota that predisposes to colorectal cancer shows values between 7 and 10.

Index of Microbiota Involvement in Favouring Intestinal Permeability (Leaky Gut)

This index is expressed in a value from 0 to 10, where 10 corresponds to an intestinal microbiota favouring intestinal permeability.

In the example, the index is based on the definition of predisposing variables (including the relative abundance of bacteria of the genera Desulfovibrionaceae and Enterobacteriaceae), and protective variables (including the potential production of butyrate) and the attribution of a “weight” to each of these variables.

The final value of the index expresses the degree of intestinal microbiota involvement in favouring intestinal permeability and is the result of a computation that takes into account the predisposing potential and the protective one of the aforesaid reference variables and the specific individual profile of relative abundance of the microorganisms involved.

Each of the variables is assigned a “weight” expressed in a value comprised between 0 and 1. For each individual considered, the value of the index of intestinal microbiota involvement in favouring intestinal permeability is obtained by adding together the “weights” of the aforesaid variables which in the individual have, in the case of the predisposing variables, a value above the 90th percentile of the corresponding distribution in healthy individuals or, in the case of the protective variable, a value below the 10th percentile of the corresponding distribution in healthy individuals.

The value obtained is then compared against the scale from 0 to 10 and shown on a graph in the form of a coloured bar in order to facilitate the reading thereof.

A normal index shows values comprised between 0 and 4, represented in a reference colour (dark grey in the example); an index of microbiota moderately favouring intestinal permeability shows values comprised between 4 and 7, shown in a further reference colour (light grey in the example); and an index of microbiota favouring intestinal permeability shows values between 7 and 10, shown in a further reference colour (black in the example).

Index of Microbiota Involvement in Favouring Systemic Aging

This index is expressed with a value from 0 to 10, where 10 corresponds to an intestinal microbiota that greatly predisposes to at least one disorder typical of aging (immunosenescence and inflammaging), whereas values close to 0 are indicative of an intestinal microbiota capable of contributing to normal immunological and/or metabolic functions of the healthy adult individual.

The index is calculated using a “Bayesian case model” and a dataset of at least 40 elderly individuals, i.e. older than 65 years age.

The index in question is based on the definition of pro-aging bacterial genera/families, in particular bacteria of the families Enterobacteriaceae, Porphyromonadaceae, Rikenellaceae, Desulfovibrionaceae, Unclassified_Clostridiales, Synergistaceae, Oscillospira and Anaerotruncus, and anti-aging genera/families, in particular the bacteria Faecalibacterium, Bifidobacterium, Roseburia, Coprococcus and Akkermansia, and on the attribution of a “weight” to each of these bacterial genera/families.

Method of Calculating the “Aging Weight” of the Reference Bacterial Groups:

Each of the bacterial genera/families selected as “pro-aging” is assigned a weight (expressed in a value comprised between 0 and 1) in terms of its contribution to the predisposition to disorders typical of aging, equivalent to the frequency with which, in a dataset of at least 40 elderly individuals, the relative abundances of said bacterial genera/families are above the 90th percentile of the corresponding distribution in the control healthy adult population.

For each “anti-aging” bacterial genus/family as well, a weight is assigned (expressed in a value comprised between 0 and 1) in terms of its contribution to the predisposition to disorders typical of aging, equivalent to the frequency with which, in the elderly individuals, the relative abundance of said bacterial genus/family is below the 10th percentile of the corresponding distribution in the control healthy adult population.

Calculation of the Maximum “Aging Weight” of the Intestinal Microbiota:

The sum of the “pro-aging” and “anti-aging” values obtained represents the maximum value that can be reached by an individual with intestinal microbiota that greatly predisposes to at least one disorder typical of the aging.

To facilitate the graphic representation and reading of the values, a mathematic proportion has been used to distribute the values of the aging index on a scale from 0 to 10, in which the maximum value is made equal to 10.

Calculation of the Individual Aging Index:

For each individual considered, the value of the aging index of the intestinal microbiota is obtained by adding together the weights of the above-described bacterial genera/families which, in the individual considered, have a relative abundance above the 90th percentile of the corresponding distribution in healthy adult individuals in the case of “pro-aging” ones, or below the 10th percentile of the corresponding distribution in healthy adult individuals in the case of “anti-aging” ones.

The value obtained is then compared against a scale from 0 to 10 and shown in the graph in the form of a coloured bar in order to facilitate the reading thereof.

A normal index shows values comprised between 0 and 4, represented in a reference colour (dark grey in the example); an index of microbiota that moderately predisposes to at least one disorder typical of aging shows values comprised between 4 and 6, represented in a further reference colour (light grey in the example); an index of microbiota that predisposes to at least one disorder typical of aging shows values comprised between 6 and 8, represented in a further reference colour (dark grey with stripes in the example); and an index of microbiota that greatly predisposes to at least one disorder typical of aging shows values comprised between 8 and 10, represented in a further reference colour (black in the example).

1. A method for determining and/or monitoring the state of health of an individual comprising the steps of:
(i) Obtaining an isolated biological sample from an individual, preferably a faecal sample or an intestinal biopsy;
(ii) Isolating the bacterial DNA from said sample;
(iii) Typing the bacterial populations of said sample, the set of said bacterial populations being likenable to the intestinal microbiota of said individual;
(iv) Assigning the typed bacterial populations to at least one taxonomic unit selected from: phylum, class, order, family, genus and species;
(v) Measuring the diversity index of said intestinal microbiota, wherein said diversity index:
a) is calculated as the number of genera and/or bacterial families typed in said sample compared to the number of genera and/or bacterial families typed in a sample of a healthy individual; and
b) is considered normal for values of the index comprised between 8 and 22, preferably 10-17;
(vi) Measuring the index of anti-inflammatory and/or immunomodulating potential of said intestinal microbiota, wherein said index:
a) is calculated on the basis of the weight or sum of weights assigned to at least one, preferably at least four, bacterial genera/families with an anti-inflammatory and/or immunomodulating potential relative to a healthy reference population, wherein, when the value of the relative abundance of said at least one bacterial genus/family falls below the 10th percentile of the reference population, the weight of that genus/family is not counted in the calculation of the index of anti-inflammatory and/or immunomodulating potential; when the relative abundance falls between the 10th and 25th percentiles of the reference population, a third of the weight calculated for said bacterial genus/family is counted; when the relative abundance falls above the 25th percentile of the reference population, the whole weight calculated for said bacterial genus/family is counted; and
b) ranges from 0 to 10 and is considered normal for values of the index comprised between 2.5 and 10;
(vii) Measuring the index of association between the intestinal microbiota of said individual and at least one disorder and/or pathological condition with metabolic and/or immunological and/or inflammatory involvement, wherein said index:
a) is calculated on the basis of the weight or sum of weights assigned to at least one bacterial genus/family associated/associable with said disorder and/or pathological condition with metabolic and/or immunological and/or inflammatory involvement having a relative abundance above the 90th percentile calculated in a reference population of healthy individuals and/or ad at least one bacterial genus/family not associated/associable with said disorder and/or pathological condition with metabolic and/or immunological and/or inflammatory involvement having a relative abundance below the 10th percentile calculated in a reference population of healthy individuals, wherein said weight ranges from 0 to 1 and, in the case of the at least one bacterial genus/family associated/associable with said disorder and/or pathological condition with metabolic and/or immunological and/or inflammatory involvement, it corresponds to the frequency with which the relative abundance of said bacterial genus/family results in a population of individuals affected by said disorder and/or pathological condition with metabolic and/or immunological and/or inflammatory involvement above the 90th percentile calculated in a reference population of healthy individuals; in the case of the at least one bacterial genus/family not associated/associable with said disorder and/or pathological condition with metabolic and/or immunological and/or inflammatory involvement, it corresponds to the frequency with which the relative abundance of said bacterial genus/family results in a population of individuals affected by said disorder and/or pathological condition with metabolic and/or immunological and/or inflammatory involvement below the 10th percentile calculated in a reference population of healthy individuals; and
b) ranges from 0 to 10 and is considered normal for values of the index comprised between 0 and 4.5;
(viii) Measuring the index of association between said intestinal microbiota of said individual and the systemic aging, wherein said index:
a) is calculated on the basis of the weight or sum of weights assigned to at least one pro-aging bacterial genus/family having a relative abundance above the 90th percentile calculated in a reference population of healthy adult individuals and/or ad at least one anti-aging bacterial genus/family having a relative abundance below 10th percentile calculated in a reference population of healthy adult individuals, wherein said weight ranges from 0 to 1 and, in the case of the at least one pro-aging bacterial genus/family, it corresponds to the frequency with which the relative abundance of said bacterial genus/family results in a population of elderly individuals above the 90th percentile calculated in a reference population of healthy adult individuals; in the case of the at least one anti-aging bacterial genus/family, it corresponds to the frequency with which the relative abundance of said bacterial genus/family results in a population of elderly individuals below the 10th percentile calculated in a reference population of healthy adult individuals; and
b) ranges from 0 to 10 and is considered normal for values of the index comprised between 0 and 6;
wherein said state of health of the individual is considered impaired if at least one of the indices according to any one of steps (v)-(viii) is considered outside the normal range.

2. The method according to claim 1, wherein the step (iii) of typing bacterial populations is carried out by amplification of at least one portion of the rRNA 16S gene and subsequent sequencing of the amplified DNA, wherein said amplification is carried out by PCR in the presence of at least one pair of primers, preferably SEQ ID NO: 1 and SEQ ID NO: 2.

3. The method according to claim 1 or 2, wherein the bacterial genera/bacterial families with an anti-inflammatory are selected from among: Faecalibacterium, Bifidobacterium, Akkermansia, Coprococcus, Lachnospira and Roseburia; more preferably Faecalibacterium, Bifidobacterium, Akkermansia and Roseburia.

4. The method according to any one of claims 1-3, wherein the disorder and/or the pathological condition with metabolic and/or immunological and/or inflammatory involvement is selected from among: obesity, type 2 diabetes, metabolic syndrome, non-alcoholic hepatic steatosis, insulin resistance, hypercholesterolaemia, deregulation of glucose metabolism, cardiovascular diseases, hypertension, Crohn's disease, ulcerative colitis, diverticular diseases, irritable bowel syndrome, allergies, food intolerances, diarrhoea, constipation, colitis and enteritis.

5. The method according to any one of claims 1-4, wherein the bacterial genus/family associated with said disorder and/or pathological condition with metabolic and/or immunological and/or inflammatory involvement is selected from among: Erysipelotrichaceae, Ruminococcus:Lachnospiraceae, Prevotella, Enterobacteriaceae, Lactobacillus, Alistipes, Collinsella, Desulfovibrionaceae, Campylobacter, Fusobacteriaceae and Megamonas; and the bacterial genus/family not associated with said disorder and/or pathological condition with metabolic and/or immunological and/or inflammatory involvement is selected from among: Faecalibacterium, Akkermansia, Bifidobacterium, Roseburia and Christensenellaceae.

6. The method according to any one of claims 1-5, wherein the pro-aging bacterial genus/family is selected from among: Enterobacteriaceae, Porphyromonadaceae, Rikenellaceae, Oscillospira, Desulfovibrionaceae, Unclassified_Clostridiales, Synergistaceae and Anaerotruncus; and the anti-aging genus/family is selected from: Faecalibacterium, Bifidobacterium, Roseburia, Coprococcus and Akkermansia.

7. The method according to any one of claims 1-6 involving a further step of measuring an index associated with intestinal permeability, wherein said index:
a) is calculated on the basis of the weight and/or sum of weights assigned to the relative abundance of at least one bacterial genus/family, selected preferably between Desulfovibrionaceae and Enterobacteriaceae, and/or based on the potential production of butyrate by the microbiota in question, calculated as the sum of relative abundances of at least one genus or bacterial family known to produce butyrate, preferably selected from among: Faecalibacterium, Roseburia, Anaerostipes, Coprococcus, Porphyromonas, Shuttleworthia, Butyrivibrio, Odoribacter and Megasphaera; and
b) ranges from 0 to 10 and is considered normal for values comprised between 0 and 7.

8. The method according to any one of claims 1-7 comprising a further step of evaluating the metabolic efficiency of said intestinal microbiota, which comprises a step of determining at least one of the following activities of said intestinal microbiota: production of acetate, production of butyrate, production of propionate, production of lactate, production of hydrogen sulphide, production of bacterial LPS (lipopolysaccharide), mucolysis or proteolysis.

9. The method according to claim 8 wherein said determination of the production of the acetate comprises a step of measuring the relative abundance in the sample under examination, compared to a reference population of healthy individuals, of at least one bacterial genus/family selected from among: Bifidobacterium, Blautia, Ruminococcus:Ruminococcaceae, Bacteroides, Odoribacter and Alistipes, wherein the value obtained is considered normal if it falls in the interval between the 10th and 90th percentiles of the reference population of healthy individuals; said determination of the production of butyrate comprises a step of measuring the relative abundance in the sample under examination, compared to a reference population of healthy individuals, of at least one bacterial genus/family selected from among: Faecalibacterium, Roseburia, Anaerostipes, Coprococcus, Porphyromonas, Shuttleworthia, Butyrivibrio, Odoribacter and Megasphaera, wherein the value obtained is considered normal if it falls in the interval between the 10th and 90th percentiles of the reference population of healthy individuals; said determination of the production of propionate comprises a step of measuring the relative abundance in the sample under examination, compared to a reference population of healthy individuals, of at least one bacterial genus/family selected from among: Bacteroides, Parabacteroides, Veillonellaceae, Coprococcus, Roseburia, Ruminococcus:Lachnospiraceae and Odoribacter, wherein the value obtained is considered normal if it falls in the interval between the 10th and 90th percentiles of the reference population of healthy individuals; said determination of the production of lactate comprises a step of measuring the relative abundance in the sample under examination, compared to a reference population of healthy individuals, of at least one bacterial genus/family selected from among: Lactobacillus, Bifidobacterium, Roseburia and Faecalibacterium, wherein the value obtained is considered normal if it falls in the interval between the 10th and 90th percentiles of the reference population of healthy individuals; said determination of the production of hydrogen sulphide comprises a step of measuring the relative abundance in the sample under examination, compared to a reference population of healthy individuals, of at least one bacterial genus/family selected from among: Bilophila, Desulfovibrio and other genera belonging to the family Desulfovibrionaceae, wherein the value obtained is considered normal if it falls in the interval between the 10th and 90th percentiles of the reference population of healthy individuals; said determination of the production of LPS comprises a step of measuring the relative abundance in the sample under examination, compared to a reference population of healthy individuals, of at least one bacterial genus/family selected from among: Enterobacteriaceae, Fusobacterium and Bacteroides, wherein the value obtained is considered normal if it falls in the interval between the 10th and 90th percentiles of the reference population of healthy individuals; said determination of the activity of mucolysis comprises a step of measuring the relative abundance in the sample under examination, compared to a reference population of healthy individuals, of at least one bacterial genus/family selected from among: Akkermansia, Bacteroides, Ruminococcus:Lachnospiraceae, wherein the value obtained is considered normal if it falls in the interval between the 10th and 90th percentiles of the reference population of healthy individuals; said determination of the activity of proteolysis comprises a step of measuring the abundance of Clostridium in the sample under examination, compared to a reference population of healthy individuals, wherein the value obtained is considered normal if it falls in the interval between the 10th and 90th percentiles of the reference population of healthy individuals.
